(19) United States
(12) Patent Application Publication
(10) Pub. No.: US 2024/0269260 A1
(43) Pub. Date: Aug. 15, 2024

(54) mRNA-Based Vaccine Composition for Inducing Immune Response Against HIV and HPV

(71) Applicants: Sarfaraz K. Niazi, Deerfield, IL (US); Matthias Magoola, Kampala (UG)

(72) Inventors: Sarfaraz K. Niazi, Deerfield, IL (US); Matthias Magoola, Kampala (UG)

(21) Appl. No.: 18/501,992

(22) Filed: Nov. 4, 2023

(51) Int. Cl. A61K 39/21; A61K 39/00; C12N 7/00

(52) U.S. Cl. CPC A61K 39/21; C12N 7/00; A61K 2039/53; A61K 2039/55555; C12N 2710/00022; C12N 2710/20034; C12N 2740/16022; C12N 2740/16034

(57) ABSTRACT

The present invention generally relates to the field of molecular biology and immunology; more specifically, the invention relates to a messenger RNA (mRNA) composition to express selected epitopes of the antigens of Human Immunodeficiency Virus (HIV) and Human Papillomavirus (HPV) for inducing an immune response to prevent or treat HIV and HPV infections.

### SEQUENCE LISTING

The instant application contains a Sequence Listing, which has been submitted electronically in ASCII format and is hereby incorporated by reference in its entirety. Said ASCII text file, created on Jan. 11, 2024, is named 27134_18501992_01-19-2024_CRFE, and is 1080 kb in size.

### BACKGROUND OF THE INVENTION

The spread of HIV and HPV poses significant health risks worldwide. Conventional vaccination strategies have had limited success in providing broad protection against these viruses due to their high mutation rates and various strains. Therefore, an improved vaccine is needed to induce a robust and broad immune response against multiple strains of HIV and HPV.

The historical progression of vaccines targeting HPV and HIV has traversed through various technological milestones. In the realm of HPV vaccines, the earliest advancements were marked by prophylactic interventions such as Gardasil and Cervarix. These relied on virus-like particles (VLPs) to trigger immune responses without the risk of infection. Subsequent innovations sought to broaden the protective spectrum of these vaccines and reduce production costs by using yeast-based VLPs. While therapeutic vaccines aimed at existing HPV infections focused on the E6 and E7 oncogenes, the potential of DNA-based and mRNA vaccines emerged more recently, leveraging these nucleic acids' capabilities to encode relevant antigens.

Nucleoside vaccines are a significant pivot from traditional approaches, as they embody the synthesis of vaccines using nucleoside-modified mRNA. This approach has risen to prominence following the success of mRNA COVID-19 vaccines, which utilize lipid nanoparticles for delivery. The inherent adaptability of nucleoside vaccines has sparked interest in their application against various pathogens, including HPV and HIV, potentially allowing for the design of vaccines that could be rapidly updated in response to viral mutations. This nucleoside-modified mRNA technology heralds a new era in vaccine development, standing on the shoulders of prior art but pushing the boundaries into novel territories of immunization strategies.

This paradigm shift towards nucleoside-modified mRNA vaccines represents a significant leap forward in immunization technology, particularly in the combat against complex viruses like HPV and HIV. The modularity of mRNA vaccines-allowing for the plug-and-play insertion of genetic codes for specific antigens-facilitates a versatile platform swiftly adapted to address emerging strains and variants, a precious characteristic given the mutagenic nature of viruses like HIV.

### SUMMARY OF THE INVENTION

The invention relates to the technical field of biotechnology, to a therapeutic HIV and HPV mRNA combination vaccine comprising a combination of multiple messenger RNA (mRNA) molecules that express conserved and immunogenic epitopes of HIV gp160 proteins P03377 ENV_HVIBR and C6G0E7_9 HIV1; and the HPV protein HPV16E7 (SOSIP.664; GenBank: ABA61516.1HPV16E6), formulated within a lipid nanoparticle (LNP) delivery system to generate higher serum antibody level and simultaneously stimulate organisms to generate TH1 and TH2 type cellular immune response against the HV and HIV viruses. The present invention is a novel composition and composition for inducing an immune response to both HIV and HPV.

### DETAILS OF THE INVENTION

To make the objects, technical solutions, and advantages of the embodiments of the present invention more apparent, the technical solutions in the embodiments of the present invention will be clearly and completely described below, but not all embodiments of the present invention. Thus, the following detailed description of the embodiments of the present invention is not intended to limit the scope of the invention, as claimed, but is merely representative of selected embodiments of the invention. All other embodiments, which can be derived by a person skilled in the art from the embodiments given herein without making any creative effort, shall fall within the protection scope of the present invention.

In summary, for HIV, a strong T cell response is crucial, mainly because of the virus's ability to evade B cell responses due to high mutation rates. On the other hand, for HPV, B cell responses leading to the production of neutralizing antibodies are very effective, especially in the context of vaccination.

For HPV, the transition to nucleoside-modified mRNA vaccines could enable the inclusion of a more comprehensive array of antigens from different HPV types, potentially offering broader protection than current vaccines. The mRNA technology also presents an opportunity for therapeutic vaccines to cure existing infections by inducing cell-mediated immunity to clear virus-infected cells.

### Human Immunodeficiency Virus (HIV)

HIV is a lentivirus that primarily infects hosts and cells through bodily fluids or pregnancy communication. HIV infects cells essential to the immune system, such as CD4+ T cells, macrophages, and dendritic cells, ultimately causing cell death. When the rate and magnitude of cell death cause essential cell levels to fall below critical levels, it becomes increasingly more difficult for the host to mount an effective immune response, leading to acquired immunodeficiency syndrome (AIDS). Without treatment, the average survival time after infection with HIV is estimated to be 9 to 11 years. In terms of HIV vaccine development, the unique challenge has been the virus's extreme genetic diversity and propensity for rapid mutation. However, the versatility of nucleoside-modified mRNA vaccines opens the door to creating multivalent vaccines that can target multiple strains of HIV simultaneously. Furthermore, as these vaccines can be produced with remarkable speed, they offer a promising solution to react quickly to the evolution of the virus within the population.

HIV has three antigen identifiers: the envelope protein gp160, the surface protein gp120, and the transmembrane protein gp41. The envelope glycoprotein is the most well-known HIV antigen. It consists of two glycoproteins, gp120 and gp41, and is responsible for the virus's ability to attach to and enter human immune cells. It's a common target for vaccines and drug development. The HIV antigens gp160 refers to a protein found on the virus's surface; it is a glycoprotein that plays a crucial role in the virus's life cycle and targets the immune system's response against HIV. It is a precursor protein that eventually cleaves into two smaller glycoproteins, gp120 and gp41. Gp120 binds to CD4 receptors on the surface of immune cells, such as T-helper cells and macrophages. This binding initiates the process of HIV entry into these cells.

The capsid (p24) protein is an HIV core protein comprising the virus's structural core. It's often used as a marker in viral load tests to measure the amount of HIV in a person's blood. The reverse transcriptase (RT) is an enzyme involved in the replication of HIV. It's targeted by some antiretroviral drugs. Integrase (IN) is another HIV enzyme targeted by certain antiretroviral medications. It plays a role in integrating the viral DNA into the host cell's genome. The protease (PR) is an enzyme that helps process viral proteins into their functional forms. Inhibition of PR is another target for antiretroviral drugs.

In contrast, the quest for an effective HIV vaccine has been challenging due to the virus's ability to evade the immune system. Initial vaccine designs attempted to stimulate neutralizing antibodies against the virus's envelope proteins. The RV144 trial underscored the necessity of inducing antibodies and cellular immune responses. This led to the exploration of broadly neutralizing antibodies (bnAbs), which some individuals naturally produce against HIV. Viral vector vaccines and nucleoside-modified mRNA platforms have been investigated, the latter being a cutting-edge approach where mRNA is used to encode HIV antigens, exploiting its ability to present these antigens in their native conformations. Both prophylactic and therapeutic strategies have been employed, with the latter seeking to control the infection in people already living with HIV.

The choice of MHC alleles for HIV infection epitope prediction considers several factors specific to the virus's immunology and the population affected by the disease. HIV mutates rapidly; therefore, identifying epitopes conserved across multiple strains can be crucial for effective vaccine design or therapeutic interventions. For HIV, CTL (Cytotoxic T Lymphocyte) responses are crucial, and these responses are mediated by peptides presented on MHC class I molecules. MHC class I alleles are associated with better control of HIV infection: HLA-B*57 and HLA-B*27. The alleles have been associated with slower progression to AIDS, suggesting that epitopes presented by these alleles could effectively elicit protective T-cell responses. HLA-B*35: This allele, in contrast, is often associated with rapid disease progression, and understanding the epitopes presented by this allele might help in understanding mechanisms of immune escape.

For HIV infection, understanding which MHC Class II alleles to focus on for epitope prediction is essential for designing effective therapies and vaccines. These alleles present antigens to CD4+T-helper cells, crucial for orchestrating the immune response, including producing antibodies and activating cytotoxic T cells. The selection of class II alleles would similarly focus on common alleles and those associated with HIV disease progression or control. Some MHC Class II alleles that have been noted in the context of HIV research include DRB1*01: This allele has been associated with slower disease progression in some studies; DRB1*03: This allele group has shown to be important in the context of HIV, though the associations can be different depending on the specific allele; DRB1*04: Some alleles in this group are also important in HIV infection and can be associated with differences in disease progression; DRB1*07: This allele has shown some protective effects in certain populations; DRB1*11: Associations with slower progression of disease have been seen with this allele in some studies; DRB1*13: This allele is also notable in some studies for its role in HIV infection; DRB1*15: This allele may have a role in influencing the immune response to HIV. DQB1 and DPB1 alleles are also part of the MHC Class II region and can present antigens to CD4+ T cells. Some specific alleles within these loci may also affect the immune response to HIV.

The most effective T-cell epitopes for P03377 ENV_HVIBR comprise No. 1: KSLEQIWNNMTW [SEQUENCE NO. 1] and No. 2 VWGIKQLQARILAVE [SEQUENCE NO. 2], or a combination thereof.

The most effective B-cell epitopes of P03377 ENV_HVIBR comprise No. 1: PNPQEVVLVNVTENFNMWKNDM [SEQUENCE NO. 3], No. 2 IRGKVQKEYAFFYKLDIIPIDND [SEQUENCE NO. 4], No. 3: TWFNSTWSTEGSNNTEGSD [SEQUENCE NO. 5], and No. 4: KQFINMWQEVGKAMYAPP [SEQUENCE NO. 6], or a combination thereof.

The most effective T-cell epitopes of C6G0E7_9 HIV1 comprise No. 1: KSLEQIWNNMTW [SEQUENCE NO. 7] and No. 2: VWGIKQLQARILAVE [SEQUENCE NO. 8], or a combination thereof.

The most effective B-cell epitopes of C6G0E7_9 HIV1 comprise No. 1: ENVTENFNMWKNDM [SEQUENCE NO. 9], No. 2: NTTVSNGSSNSNANFEEM [SEQUENCE NO. 10], No. 3: EHFPNRNITFNHSSGGDL [SEQUENCE NO. 11], No. 4: HPNGTYNETAVNSSD [SEQUENCE NO. 12], No. 5: NMWQEVGRAMYAPP [SEQUENCE NO. 13], No. 6: QEKNEKDLLALDSWQN [SEQUENCE NO. 14], and No. 7: IKDKKKNEYALFYKL [SEQUENCE NO. 15] or a combination thereof.

### Human Papillomavirus (HPV)

HPV infection is a significant factor in cervical intraepithelial neoplasia and cervical cancer development. In recent years, the prevalence and mortality of cervical cancer in our country have gradually increased and tended to be younger. About forty thousand patients with cervical cancer in China currently have a mortality rate of 11.30 percent, which is far higher than the mortality rate of 5 percent in developed countries. Among them, 50% of cervical cancers are associated with HPV16 infection, which is a high-risk type of HPV that is strongly associated with cervical cancer and other cancers in the genital and oropharyngeal regions. It produces several proteins, the most important ones being E6 and E7. The E6 protein is known for binding to and deleting the tumor suppressor protein p53. This interaction can lead to the uncontrolled growth of infected cells and is a critical step in the development of HPV-related cancers. The HPV16 E7 protein also plays a role in promoting cell proliferation and inhibiting cell differentiation. It interacts with various cellular proteins, including the retinoblastoma (Rb) tumor suppressor protein, disrupting normal cell cycle regulation. The HPV17 is a relatively low-risk type of HPV compared to HPV16. It is not as strongly associated with cancer, but it can still cause genital warts. Detailed information about specific proteins related to HPV17 may be limited because it has received less research attention than high-risk HPV types like HPV16. The present invention aims to provide an mRNA molecule that can independently express multiple antigens in vivo, taking full advantage of mRNA molecules over protein and DNA molecules.

Cell-mediated immune responses, rather than humoral immunity, are essential for clearing HPV infections and killing tumor cells. Studies have shown that spontaneous clearance of HPV infection and persistent HPV infection is associated with a strong cell-mediated immune response in the body, mainly involving TH 1-type cells and the induction of cytotoxic T lymphocytes from CD4+ and CD8+ T cells, respectively. However, the pre-existing immune response to viral vector vaccines remains challenging, and viral genes risk integration into the host genome.

The identification of T cell epitopes for HPV is crucial for the development of therapeutic vaccines, as prophylactic vaccines are already available (e.g., Gardasil and Cervarix) that target the L1 protein of the virus but do not have therapeutic effects against established infections. For HPV, the following considerations can be made when selecting MHC alleles for epitope prediction. MHC Class I Alleles may include HLA-A*02: This is one of the most common alleles worldwide and is known to present HPV peptides and extensively studied in the context of HPV; HLA-B and HLA-C alleles: Depending on your target population, other HLA-B or HLA-C alleles may also be relevant. MHC Class II Alleles: Helper T cell responses are crucial for orchestrating the immune response and the help provided to CTLs. MHC class II molecules present epitopes to CD4+ T cells; DRB1*15:01 and DRB1*04 are examples of standard MHC class II alleles that might be of interest. They have been associated with HPV persistence and cervical cancer risk in certain populations.

The most effective T-cell epitopes of HPV16E7 (SOSIP.664) (GenBank: ABA61516.1HPV16E6) comprise No. 1: NNTIIRFANSSGGD [SEQUENCE NO. 16], No. 2: GNNTIIRFANSSGGD [SEQUENCE NO. 17], No. 3: NNTIIRFANSSGG [SEQUENCE NO. 18], No. 4: FGNNTIIRFANSSGGD [SEQUENCE NO. 19], No. 5: GNNTIIRFANSSGGDL [SEQUENCE NO. 20], No. 6: [SEQ. NO. 31], or a combination thereof.

The most effective B-cell epitopes of HPV16E7 (SOSIP.664) (GenBank: ABA61516.1HPV16E6) comprise No. 1: ENVTEEFNMWKNN [SEQUENCE NO. 21], No. 2: TNVTNNITDDMRGE [SEQUENCE NO. 22], No. 3: ELRDKKQKVYSLFYRL [SEQUENCE NO. 23], No. 4: VVQINENQGNRSNNSN [SEQUENCE NO. 24], No. 5: AITQACPKVSFEPIP [SEQUENCE NO. 25], No. 6: TWISNTSVQGSNSTGSND [SEQUENCE NO. 26], No. 7: KQIINMWQRIGQAMYAPPIQ [SEQUENCE NO. 27], No. 8: VVKIEPLGVAPTRAKRRVVGREK [SEQUENCE NO. 28], No. 9: PWNSSWSNRNLSEIWDNMT [SEQUENCE NO. 29], and No. 10: EKNEQDLLALDKWASL [SEQUENCE NO. 30], or a combination thereof.

Messenger RNA (mRNA)

mRNA molecules are designed to encode for specific antigens of HIV and HPV. The mRNA is structurally composed of a 5′UTR, a signal peptide for efficient translation, the open reading frame that encodes the antigen, a 3′UTR for stability, and a polyA tail. This structure ensures efficient translation and antigen presentation for an immune response. Using pseudouridine in the mRNA is a modification to avoid innate immune sensing and enhance translation efficiency. An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs. However, unlike the ORF, those elements need not necessarily be present in a vaccine of the present disclosure.

The mRNA vaccine platform is safe, does not infect the body or integrate into the genome, and does not carry the risk of infection or mutation. Its immune effect is better and better stabilized by various modifications and encapsulation, yielding strong humoral and cellular immune responses. It is also most convenient to produce at a low cost, making the mRNA vaccine a significant humanitarian assistance. The mRNA vaccine can be produced rapidly on a large scale by an in vitro transcription technology, and compared with the traditional vaccine with a production period of 5-6 months or longer, the production preparation of the mRNA vaccine sample can be completed within dozens of days. The outburst epidemic situation can be dealt with more efficiently.

After the mRNA vaccine containing the mRNA molecule is used for immunization, an organism can simultaneously generate humoral and cellular immune responses aiming at a plurality of antigens in an induced mode. Therefore, a better immune effect is achieved. Thus, the claimed invention comprises a mixture of multiple mRNA molecules, each capable of expressing one antigenic protein, two for HIV and one for HPV infection immunity generation.

The nucleoside modifications within the mRNA are crucial because they help to evade the host's innate immune responses, which can often degrade mRNA before it achieves its purpose. These modifications can enhance the translational capacity and stability of the mRNA, leading to higher and more prolonged protein expression of the vaccine antigen within the body. As a result, these vaccines can induce robust and sustained immune responses, which are critical for preventive and therapeutic vaccine strategies.

### Lipid Nanoparticle (LNP)

The LNP delivery system is an essential component of the invention, allowing for the encapsulation and delivery of the mRNA into human cells. The LNPs are designed to include an ionizable cationic lipid for effective delivery, a non-cationic lipid for structural stability, and a PEGylated lipid to extend circulation time in the bloodstream. Lyophilization of the LNP is included to enhance stability and shelf-life, making the vaccine suitable for distribution and storage.

In some respects, the present disclosure provides compositions of inducing in a human subject an immune response to HIV, the compositions comprising administering to the subject a lipid nanoparticle comprising a mRNA encoding epitopes of HIV gp160 proteins P03377 ENV_HVIBR, and C6G0E7_9 HIV1; and HPV proteins HPV16E7 (SOSIP.664; GenBank: ABA61516.1HPV16E6).

Lyophilization of LNP formulation extends the storage life of the product. It allows storage at higher temperatures, making it an essential consideration for the distribution of the HIV and HPV vaccines that are direly needed in many countries where storage conditions are non-compatible for non-lyophilized products.

### EMBODIMENTS

In the first embodiment, the present invention uses multiple mRNA molecules to provide a vaccine to prevent or treat HIV and HPV.

In the second embodiment, the present invention provides mRNA molecules, each expressing selected T-cell and B-cell epitopes of HIV gp160 protein (UniProt: P03377 ENV_HVIBR and UniProt: C6G0E7_9 HIV1); and HPV protein HPV16E7 (SOSIP.664; GenBank: ABA61516.1), la glycoprotein trimer designed to mimic the structure of the HIV envelope protein found on the surface of the virus. The “SOSIP” in SOSIP.664 stands for “stabilized, soluble, cleaved, trimeric” and refers to the modifications made to the protein to make it more stable and mimic the native viral spike.

In a third embodiment, the present invention provides a therapeutic mRNA molecule comprising a 5′UTR element, a signal peptide element, an open reading frame corresponding to the expressed proteins, a 3′ UTR element, and a polyA tail element linked together in that order.

In a fourth embodiment, the uracil, cytosine, or adenine nucleotides of the therapeutic mRNA molecule contain a modifying group that includes at least one of pseudouridine, N1-methyl pseudouridine, N1-ethylpseudouridine, 5-methylcytosine, 2-thiouridine, 5-methoxyuridine, or N1-methyladenosine. RNA sequences are modified by replacing uridine (U) with pseudouridine (Y).

In a fifth embodiment, the present invention claims an LNP formulation that comprises the steps of mixing D-Lin-MC3-DMA, DSPC, cholesterol, and DMG-PEG 2000 in an absolute ethanol solution, adding the mixture into a citrate buffer solution, and extruding the mixture by a liposome extruder to obtain the liposome nanoparticle.

1. A composition for inducing in a human subject an immune response to Human Immunodeficiency Virus (HIV) and Human Papillomavirus (HPV), the composition comprising messenger RNA (mRNA) molecules expressing T-cell and B-cell epitopes of HIV proteins gp160 (P03377 ENV_HVIBR and C6G0E7_9 HIV1), and HPV protein HPV16E7; SOSIP.664; GenBank: ABA61516.1HPV16E6), formulated in a lipid nanoparticle.

2. The composition of claim 1, wherein the selected T-cell epitopes of P03377 ENV_HVIBR comprise SEQ. NO. 1 and SEQ. NO. 2, or a combination thereof.

3. The composition of claim 1, wherein the selected B-cell epitopes of P03377 ENV_HVIBR comprise SEQ. NO. 3, SEQ. NO. 4, SEQ. NO. 5 and SEQ. NO. 6, or a combination thereof.

4. The composition of claim 1, wherein the selected T-cell epitopes of C6G0E7_9 HIV1 comprise SEQ. NO. 7, and SEQ. NO. 8, or a combination thereof.

5. The composition of claim 2, wherein the selected B-cell epitopes of C6G0E7_9 HIV1 comprise SEQ. NO. 9, SEQ. NO. 10, SEQ. NO. 12, SEQ. NO. 12, SEQ. NO. 13, SEQ. NO. 14 and SEQ. NO. 15, or a combination thereof.

6. The composition of claim 1, wherein the selected T-cell epitopes of HPV16E7 (SOSIP.664; GenBank: ABA61516.1HPV16E6) comprise SEQ. NO. 16, SEQ. NO. 17, SEQ. NO. 18, SEQ No. 19, SEQ No. 20, and SEQ No. 31 or a combination thereof.

7. The composition of claim 1, wherein the selected B-cell epitopes of HPV16E7 (SOSIP.664; GenBank: ABA61516.1HPV16E6) comprise SEQ. NO. 21, SEQ. NO. 22, SEQ. NO. 23, SEQ. NO. 24, SEQ. NO. 25, SEQ. NO. 26, SEQ. NO. 27, SEQ. NO. 28, SEQ. NO. 29, and SEQ. NO. 30, or a combination thereof.

8. The composition of claim 1, wherein the mRNA molecules comprise a 5′UTR element, a signal peptide element, an open reading frame corresponding to the expressed proteins, a 3′ UTR element, and a polyA tail linked together in that order.

9. The composition of claim 8, wherein the open reading frame of the mRNA molecules comprises nucleosides to express epitopes selected epitopes of HIV gp160 protein (P03377 ENV_HVIBR and C6G0E7_9 HIV1), and HPV16E7 (SOSIP.664; GenBank: ABA61516.1HPV16E6) protein.

10. The composition of claim 8, wherein RNA sequences are modified by replacing uridine (U) with pseudouridine (Y), wherever applicable, in the mRNA sequence.

11. The composition of claim 1, wherein the mRNA molecules are enclosed in lipid nanoparticles further comprising an ionizable cationic lipid, a non-cationic lipid, and a PEG-modified lipid.

12. The composition of claim 1, wherein the lipid nanoparticles are lyophilized.

13. The composition of claim 11, wherein the lipid nanoparticle formulation comprises the steps of mixing D-Lin-MC3-DMA, DSPC, cholesterol, and DMG-PEG 2000 in an absolute ethanol solution, adding the mixture into a citrate buffer solution, and extruding the mixture by a liposome extruder to obtain the liposome nanoparticle.


SEQUENCE LISTING (31 sequences)

SEQ ID NO: 1 - protein, 12 aa, Human immunodeficiency virus 1
KSLEQIWNNMTW

SEQ ID NO: 2 - protein, 15 aa, Human immunodeficiency virus 1
VWGIKQLQARILAVE

SEQ ID NO: 3 - protein, 22 aa, Human immunodeficiency virus 1
PNPQEVVLVNVTENFNMWKNDM

SEQ ID NO: 4 - protein, 23 aa, Human immunodeficiency virus 1
IRGKVQKEYAFFYKLDIIPIDND

SEQ ID NO: 5 - protein, 19 aa, Human immunodeficiency virus 1
TWFNSTWSTEGSNNTEGSD

SEQ ID NO: 6 - protein, 18 aa, Human immunodeficiency virus 1
KQFINMWQEVGKAMYAPP

SEQ ID NO: 7 - protein, 12 aa, Human immunodeficiency virus 2
KSLEQIWNNMTW

SEQ ID NO: 8 - protein, 15 aa, Human immunodeficiency virus 2
VWGIKQLQARILAVE

SEQ ID NO: 9 - protein, 14 aa, Human immunodeficiency virus 2
ENVTENFNMWKNDM

SEQ ID NO: 10 - protein, 18 aa, Human immunodeficiency virus 2
NTTVSNGSSNSNANFEEM

SEQ ID NO: 11 - protein, 18 aa, Human immunodeficiency virus 2
EHFPNRNITFNHSSGGDL

SEQ ID NO: 12 - protein, 15 aa, Human immunodeficiency virus 2
HPNGTYNETAVNSSD

SEQ ID NO: 13 - protein, 14 aa, Human immunodeficiency virus 2
NMWQEVGRAMYAPP

SEQ ID NO: 14 - protein, 16 aa, Human immunodeficiency virus 2
QEKNEKDLLALDSWQN

SEQ ID NO: 15 - protein, 15 aa, Human Papillomavirus 1
IKDKKKNEYALFYKL

SEQ ID NO: 16 - protein, 14 aa, Human Papillomavirus 1
NNTIIRFANSSGGD

SEQ ID NO: 17 - protein, 15 aa, Human Papillomavirus 1
GNNTIIRFANSSGGD

SEQ ID NO: 18 - protein, 13 aa, Human Papillomavirus 1
NNTIIRFANSSGG

SEQ ID NO: 19 - protein, 16 aa, Human Papillomavirus 1
FGNNTIIRFANSSGGD

SEQ ID NO: 20 - protein, 16 aa, Human Papillomavirus 1
GNNTIIRFANSSGGDL

SEQ ID NO: 21 - protein, 13 aa, Human Papillomavirus 1
ENVTEEFNMWKNN

SEQ ID NO: 22 - protein, 14 aa, Human Papillomavirus 1
TNVTNNITDDMRGE

SEQ ID NO: 23 - protein, 16 aa, Human Papillomavirus 1
ELRDKKQKVYSLFYRL

SEQ ID NO: 24 - protein, 16 aa, Human Papillomavirus 1
VVQINENQGNRSNNSN

SEQ ID NO: 25 - protein, 15 aa, Human Papillomavirus 1
AITQACPKVSFEPIP

SEQ ID NO: 26 - protein, 18 aa, Human Papillomavirus 1
TWISNTSVQGSNSTGSND

SEQ ID NO: 27 - protein, 20 aa, Human Papillomavirus 1
KQIINMWQRIGQAMYAPPIQ

SEQ ID NO: 28 - protein, 23 aa, Human Papillomavirus 1
VVKIEPLGVAPTRAKRRVVGREK

SEQ ID NO: 29 - protein, 19 aa, Human Papillomavirus 1
PWNSSWSNRNLSEIWDNMT

SEQ ID NO: 30 - protein, 16 aa, Human Papillomavirus 1
EKNEQDLLALDKWASL

SEQ ID NO: 31 - protein, 15 aa, Human Papillomavirus 1
NNTIIRFANSSGGDL